(19) United States
(12) Patent Application Publication
(10) Pub. No.: US 2003/0235876 A1
(43) Pub. Date: Dec. 25, 2003

(54) Novel Bartonella antigen lysate extracts for use in ELISA diagnostic

(76) Inventor: Frederick Randall Bethke, Millsboro, DE (US)

Correspondence Address: INTERVET INC, 405 STATE STREET, PO BOX 318, MILLSBORO, DE, 19966, US

(21) Appl. No.: 10/176,735

(22) Filed: Jun. 21, 2002

(51) Int. Cl. G01N 33/554; G01N 33/569; C12N 9/00

(52) U.S. Cl. 435/7.32; 435/183; 435/69.3

(57) ABSTRACT

Embodiments of the present invention generally relate to a novel bacterial antigen extraction and methods for its use. Further embodiments of the present invention use the novel bacterial antigens in diagnostic kits for the detection of Bartonella antibodies in serum.

### FIELD OF THE INVENTION

The present invention relates to processes and kits for the detection of Bartonella in and organism.

### BACKGROUND OF THE INVENTION

Conventional immunoassays for detecting antibodies and/or antigens include enzyme immunoassays such as the ELISA (enzyme-linked immunosorbent assay) protocol, radioimmunoassays such as the RIA-immunoprecipitation assay, and immunofluorescence protocols. Typically, a predetermined quantity of antigen (or antibody) is adsorbed on a solid phase, protein-binding surface. The test sample to be assayed for antibodies (antigens) is then contacted to the surface having antigen (antibody) bound thereto, and antibodies (antigens) in the test sample bind to the immobilized antigen (antibody). Radioactive or enzyme-labeled immunoglobulin probes are then contacted to the surface and bind to the immobilized antibodies (antigens). The amount of labeled probe bound to the solid support can be quantitated and is indicative of the antibody (antigen) concentration in the test sample.

Disadvantages of using radioimmunoassay procedures include the necessity of extensive sample manipulations, including multiple dilutions, incubations and washing steps. In addition, potentially hazardous radioisotopes are employed. Processing samples according to a radioimmunoassay protocol consumes at least several hours, and requires relatively complex laboratory equipment and skilled technicians.

On the other hand, immunofluorescent staining generally provides an accurate indication of specificity, and it permits visualization of the antigen-antibody reaction. Immunofluorescence methodologies, however, are time consuming and difficult to perform on a large scale. Moreover, analysis of immunofluorescence assay results requires the analytical judgment of experienced technicians. Moreover, sensitivity and interference from ions are issues encountered in immunofluorescence assays.

Another type of immunoassay includes enzyme-linked immunosorbent assays (ELISA). ELISA protocols typically involve multiple microassays utilizing several dilutions of serum and a single target antigen (antibody) concentration. Microtiter plates are typically used for performing the multiple microassays necessary to detect the presence of antibody (antigen). ELISA multi-well techniques have the following procedural similarities:

1. Wells of polystyrene micro-titer plates are sensitized by passive absorption with the relevant antigen; the plates are then washed.

2. The test samples are incubated in the sensitized well and the plates are again washed. Antibody present in that sample is bound to the immobilized antigen on the well surfaces.

3. Enzyme-labeled anti-Ig (i.e., anti-immunoglobulin antibody to the animal species corresponding to the sample) conjugate is incubated in the wells. The conjugate contains an enzyme such as horseradish peroxidase, glucose oxidase, beta-galactosidase or alkaline phosphatase. The conjugate reacts with any “captured” or bound antibody. Excess reagent is washed away.

4. Enzyme substrate is added and the plates are incubated; the rate of degradation is indicated by a color change, which is proportional to the antibody concentration in the test samples in Step 2.

5. The reaction is stopped or allowed to arrest and the color change is assessed visually or in a spectrophotometer.

Such ELISA procedures are useful but commonly are not as specific as other immunoassay techniques. In particular, ELISA OMP processes for the detection of Bartonella tend not to be specific at low levels.

Bartonella henselae is an agent of human cat scratch disease (CSD) and has been associated with bacillary angiomatosis, bacillary peliosis, recurrent bacterimia, and endocarditis. (cat scratch disease, bacillary angiomatosis, and other infections due to Rochalimaea N., et al., New England Journal of Medicine, 1994, 330: pages 1509-1515). While cats have been shown through evidence to serve as vectors for the transmission of Bartonella henselae to people, cats appear to be asymptomatic to natural infection. (Bartonella R. Bacteriamia and Three Feline Populations, Kordick et al., abstracts of the 34 Inter Science Conference on Anti-Microbial Agents and Chemotherapy, American Society for Microbiology Washington D.C., 1994). However, some recent studies have indicated that experimentally infected cats may develop clinical signs such as fever, anorexia, lethargy, and peripheral lymphadenopathy. (Experimental and Natural Infection with Bartonella henselae in domestic cats, et al., Comp. Immuno. Microbial. In Fact. Dis., 1997, 20:pages 41-51). These clinical signs dissipate within a short time and may not even be noticed by the cat owner. However, infections are prone to relapse.

It has been well documented in the literature that there is a strong immune response to infection with Bartonella henselae. (Identification of Bartonella-specific imunodominant antigens recognized by the feline humoral immune system, Freeland et al, Clinical and Diagnostic Immunology, July 1999, pages 558-566). However, the parthogenesis of Bartonella henselae in cats is not clearly understood. A complicating factor in the detection of Bartonella henselae is that cats naturally infected with Bartonella henselae commonly have periods of recurring bacteremia that may last months to years without causing clinical disease during those periods. (Clinical disease in kittens inoculated with the pathogenic strain of Bartonella henselae, Mikolajczyk et al., AJVR, volume 61, Number 4, April 2000, page 375).

There are conflicting reports with regard to clinical signs of experimentally infected cats. (Experimental of Natural infection with Bartonella henselae in domestic cats, Abbott et al., Comp. Immunol. Microbol. Infect. Dis., 1997 20: pages 41-51). Various studies have reported absence of clinical signs in experimentally infected cats while others have reported mild clinical signs, including mild fever, as well as histopathological lesions in some cats up to 8 weeks post infection. (Relapsing bacteremia after blood transmission of Bartonella henselae infected cats, Kordick et al., American Journal of Veterinary Research, 1997, 58: pages 492-497). Other clinical signs in kittens experimentally infected with Bartonella henselae have included lethargy and anorexia. (Clinical disease in kittens inoculated with the pathogenic strain of Bartonella henselae, Mikolajczyk et al., AJVR, volume 61, Number 4, April 2000, page 378). Another interesting observation is that kittens infected with Bartonella henselae have experienced two episodes of clinical signs as opposed to adult cats infected with Bartonella henselae having experienced only one episode of clinical signs. (Id).

Pet cats are not normally screened for Bartonella infections or for antibodies to B. henselae. However, serological screening could be beneficial to owners who are immunocompromised or to owners having young children by safeguarding against the adoption of potentially infected cats. Cat scratch disease can lead to potentially serious diseases in humans, particularly in young children and immunocompromised individuals. Therefore, screening of cats for Bartonella infections is desirable.

Of the commercially available diagnostic tools discussed above, the most common is immunofluorescence assays (IFAs). (Response to “Rochalimaea henselae” antigens in suspected cat scratch disease, Regnery et al., Lancet, 1992, 339:pages 1443-1445). IFA has a number of limitations. The assay is difficult to perform with large numbers of samples, is time consuming, is costly, and requires microscopes with fluorescent light sources. The other commonly used immunoassay, ELISA, tends to be both. Accordingly, the art field is in search of a process and/or diagnostic kit for the detection of antibodies to Bartonella that is relatively easy to operate, simple, and non-hazardous. The present invention, in an embodiment, provides such a process and diagnostic kit.

### SUMMARY OF THE INVENTION

Embodiments of the present invention provide a process and diagnostic kit for the accurate, rapid and sensitive assay of antibody responses to Bartonella infection in an organism.

Various other embodiments of the present invention provide an ELISA diagnostic kit for Bartonella infection to be used while remaining in the field. The novelty and originality of the ELISA diagnostic kit of the present invention at least partially resides in the particular combination of a novel purification method of the antigen to be used and the novel methods of the associated kits.

Generally, embodiments of the present invention utilize a soluble fraction of a bacterial antigen extraction as a coating antigen in a solid phase of an enzyme-linked immunosorbent assay (ELISA) whereas the prior art antigen is commonly derived from the insoluble or pelleted fraction, the outer membrane protein (hereinafter referred to as “OMP”). Embodiments of the present invention demonstrate enhanced sensitivity and more species-specific reaction as compared to the prior art immunodiagnostic techniques.

Further aspects of various embodiments of the present invention include centrifuges, sonicators and/or absorbance readers for determination of the antibodies in the sample.

### BRIEF DESCRIPTION OF THE DRAWING

FIG. 1 is an illustration of an embodiment of a plate for use in a process and/or diagnostic kit of the present invention.

FIG. 2 is an illustration of a bound antigen to an embodiment of a plate for use in a process and/or diagnostic kit of the present invention.

### DETAILED DESCRIPTION OF THE INVENTION

As used herein, the term “harvest” and any conjugation thereof means and refers to collect or collection. As used herein, the term “in a volume sufficient to coat” means and refers to a volume sufficient to provide sufficient binding moieties to react with at least substantially all bound antigens (or antibodies, as the case may be).

Embodiments of the present invention provide a novel lysate of a Bartonella antigen from Bartonella bacteria cells. Further embodiments describe a novel process for extracting the lysate from the Bartonella bacteria cells. Other embodiments utilize the novel lysate in diagnostic kits and/or immunoassays for the detection of antibodies to Bartonella in a sample, such as a serum sample.

Embodiments of the present invention can be used for preparation of lysates for all types of Bartonella, including, but not limited to, Bartonella henselae, Bartonella quintana, Bartonella bacilliformis, Bartonella vinsonii, Bartonella clarridgeiae and the like.

Embodiments of the present invention for preparing a lysate generally comprise a novel process for extracting Bartonella antigen, comprising the steps of:

(a) harvesting Bartonella bacteria cells;

(b) separating the Bartonella bacteria cells;

(c) sonicating the suspended Bartonella bacteria cells; and,

(d) extracting the soluble fraction.

Embodiments of the extracted soluble fraction, the lysate or supernatant, of the present invention may be utilized in an immunoassay, such as an enzyme-linked immunoassay, such as an enzyme-linked immunosorbent assay (ELISA).

Harvesting the Bacteria:

The bacteria may be obtained from numerous sources. In various embodiments, the bacterial antigen is extracted from a cell, such as a bacteria cell. The methods known to isolate or extract antigens from cells are quite varied. Because of considerable dissimilarity of components of different bacterial species, methods of wide application are few. Bacterial antigens may be: 1) extracellular such as extracellular proteins, flagella and exopolysaccharides; 2) part of the cell wall; 3) part of the cell membrane; and/or 4) intracellular components. An antigen, however, that can be obtained from a suspension of purified antigen will be almost free from contaminating host material. This is likely to provide an antigen free of other antigens and most interfering matter.

In an embodiment, the Bartonella is grown on blood agar flasks. In an embodiment, the Bartonella is grown in an artificial environment. The artificial environment can be created by varying the concentration of some or all of the constituents of the atmosphere about the Bartonella, such as varying the temperature, and/or the like.

The Bartonella grown may be harvested by any method common in the art, such as by scraping with a spatula. In another embodiment, cells are harvested with glass beads.

After harvesting the bacteria, the bacterial antigen is extracted.

Extraction of the Antigen:

The extraction of the antigen from the Bartonella of the present invention retains the antigen in the soluble fraction, the supernatant. In an embodiment, the extraction is performed by a first separation of the Bartonella cells; a first suspension of the cells in a saline solution; sonicating the suspension; separating the sonicated suspension; and, extracting the supernatant. These steps may be performed by manners common in the art. Moreover, various embodiments of the present invention may include the steps of a second separation of the Bartonella cells and/or a second suspension of the Bartonella cells in a detergent buffer. Other embodiments may not perform all the above recited steps in extracting the antigen. Such recited steps are exemplary and for illustration only and not mandatory.

In an embodiment, the Bartonella cell(s) are separated in a first separation by centrifugation. In such an embodiment, the cells may be centrifuged for a time sufficient to form a pellet. By forming a pellet is meant that a sample is separated based upon sedimentation properties. The pellet of a separation is typically the portion with a greater density. The pellet portion may be defined by solid portion and/or defined as a portion of the separation. The pellet, at this stage of the process, may contain at least a portion of the antigens of the cell(s). However, in other embodiments, the pellet contains no antigens of the cell at this stage of the process. Other embodiments may utilize other methods of separation, as the method of separation is not critical, such as homogenation, presses, Nitrogen cavitation, osmotic separation, hypotonic separation, sonication, freeze-thaw, detergent separation, chaotropic agents, enzymatic processes, and/or the like.

After the Bartonella is separated in a first separation, it is suspended in a first suspension. In an embodiment, the first suspension of the separated cells is in a saline solution, such as a Borate saline solution at pH 9.0. However, other saline solutions can be used, such as, by way of example and not as a limitation, phosphate buffered saline, tris buffered saline and the like. As well, the pH of the saline solution can be varied, but, for best results, a more basic solution should be used. After the first suspension, the first suspension may be vortexed, or swirled. Vortexing the solution will assist in uniform dispersion of bacterial cell clumps.

The suspended solution is separated once more in a second separation, such as by centrifugation or the like as above. The supernatant is poured off, disposed of, and the pellet and/or pellet portion retained for suspension in a second suspension. In a preferred embodiment, the second suspension is in a non-ionic detergent solution. The detergent solution extracts at least a portion of the antigen from the pellet into the supernatant.

In certain embodiments, the second suspension is then disrupted to further separate clumping cells. In preferred embodiments, the disruption is performed with a sonicator, such as a Branson Model 450 Sonifier. In a preferred embodiment, the suspension is cooled in an ice bath during sonication to prevent excessive heating of the solution. Such devices as a water-cooled cup-horn may optionally be used to cool the suspension. The disrupted solution may then be separated in a third separation, such as by centrifugation as above. However, the supernatant of this separation is retained whereas the pellet is discarded.

Included within the scope of the present invention are processes that include both fewer and/or more steps for separation of the antigen of the Bartonella into the soluble fraction, the supernatant. For example, processes including multiple steps of separating and/or multiple steps of suspending may be used.

In various embodiments, the supernatant is aliquoted into separate samples. These separate samples may be frozen and/or stored otherwise until needed. Freezing and/or storing of the sample has not been shown to adversely affect the lysate of the present invention. The lysate of the present invention has been found to be extraordinarily stable and can last for periods of at least 4 years, in certain embodiments, without experiencing sample degradation.

The extracted antigen is then ready for use in an immunoassay or as desired otherwise.

Use of the Lysate in an Immunoassay:

Various immunoassay(s) may be used with the lysate of the present invention, such as enzyme-linked immuno assay, immunofluorescence assay, radioimmunoassays, and the like. In a preferred embodiment, the immunoassay is an enzyme-linked immunosorbent (ELISA) assay for measuring humoral immune (antibody) responses to the Bartonella extract.

ELISAs generally only require basic equipment. Typically, but not exclusively, ELISAs require a plate upon which to bind the antigen (or antibody), reagents, a sample (such as a serum sample), a secondary antibody which is linked to an enzyme label and can bind to the bound antibody in the above step, an enzyme substrate, and a spectrophotometric reader. However, it is understood that ELISA methods are diverse and that ELISA methods utilizing more or less equipment may be used with embodiments of the present invention.

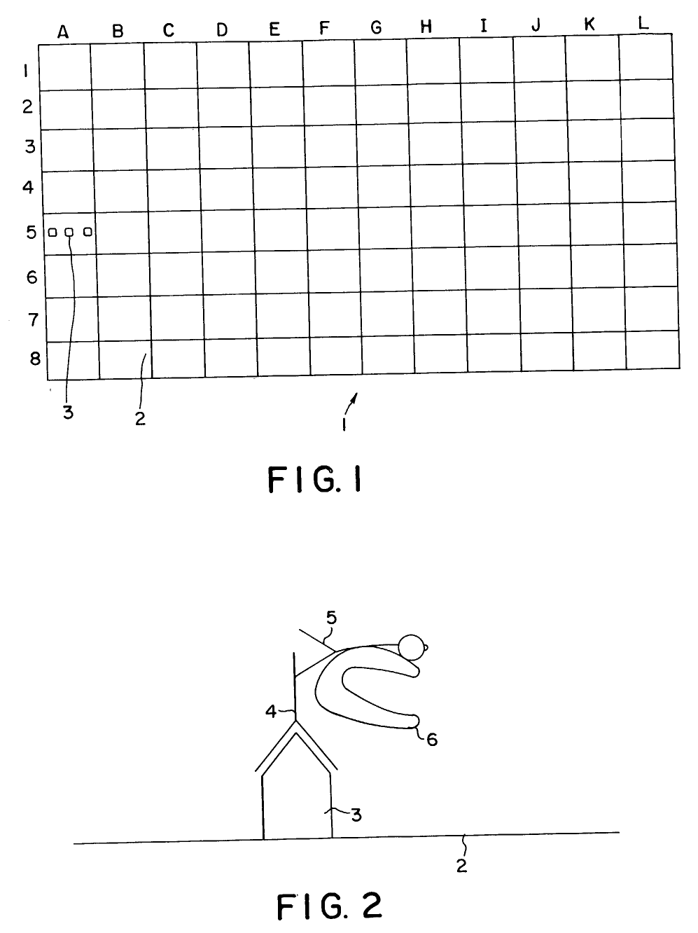

A suitable plate and/or tray which may be used in carrying out an immunoassay of the present invention is illustrated in FIG. 1. This exemplary chambered plate 1, in this embodiment twelve chambered, plate is available commercially from 12-chambered plate. The chambers are commonly referred to as wells 2. Desirably the plate is divided into a plurality of identifiable wells. For example, the tray may be marked on the bottom side to indicate zones, in this case, 96 zones. This can be accomplished by labeling the twelve cells “A”, “B”, “C”, “D” and so on, at approximately equal distances vertically, labeling the numbers “1”, “2”, “3”, “4” and so on, on plate 1. The 48 zones, of this embodiment, are thus denoted as cell A-1, cell A-2, etc. Wells 2 may be cleaned, as is common in the art. Alternatively, petri dishes, multi-well microtitration plates, and the like may be used.

For Bartonella antigens, a source of Bartonella antigen, such as B. henselae antigen is added in a dilute solution of concentrations of about 0.5 to 10 μg protein or lipopolysaccharide per milliliter to each cell of a plate and incubated for sufficient time to cause the antigen to become attached to the tray surface. However, other embodiments may not dilute the antigen solution.

Reference to FIG. 1 illustrates a well 2 of a plate 1 with bound antigen 3. Other binding solutions that may be used include Bartonella and a buffer, such as a saline solution, for binding optimal concentrations of the antigen to decrease background binding and false positives in an analysis. Many antigens may be incubated for about 2 to 4 hours at room temperature to effect binding. Other antigens preferentially bind at different conditions, such as 3 hours at 37° C. followed by overnight at 4° C., or other combination of temperatures and/or time.

In preferred embodiments, plate 1 is washed with a buffer solution, such as PBS with detergent such as tween-20 and a preservative such as thimerosal. However, in other embodiments, plate 1 may not be washed with a buffer before coating. An ELISA diluent, comprising Tween-20 and 5% Carnation non-fat skim milk can be used as an ELISA diluent, in a volume sufficient to coat. After binding of the antigen, the excess solution is then discarded and the tray compartments, now bearing attached antigen are refilled with a solution of a blocking agent, such as albumin, non-fat milk, ovalbumin, gelatin, serum, and the like, for the purpose of attaching an inert material to plastic binding sites which were left exposed after the incubation with antigens. This step reduces the non-specific adsorption of antibody molecules which are not directed at the specific antigen and reduces non-specific adsorption of the conjugate which is important in the colorimetric steps. The solution is incubated for another 45 minutes at room temperature and discarded.

A serum sample is then added, in a volume sufficient to coat, to at least one well at a concentration. In an embodiment, the concentration is known. In a preferred embodiment, multiple concentrations are used, such as by serially diluting the serum sample on the ELISA plate 1. In an embodiment, on plate 1, 12 samples (A-L) can be run. Each sample could be serially diluted, for example, from 1:1K dilution to 1:128K dilution (i.e. 1, 2, 4, 8, 16, 32, 64, 128). Plate 1 is allowed to incubate for an amount of time sufficient for at least some binding of antibodies 4 in the serum sample to bound antigen 3 (referring to FIG. 2). In a preferred embodiment, the serum was incubated in wells 2 for approximately 1 hour at about 37° C.

After incubation the serum and diluent are poured off and the wells washed sufficiently to remove serum remaining in any of wells 2. In preferred embodiments, plate 1 is washed at least 4 times with a buffer solution, such as PBS.

Next, a conjugate in the form of a species-specific enzyme linked anti-immunoglobulin is applied to the test plate. Conjugates are commercially available. Most are made in the goat or rabbit, however other conjugates common in the art may be used. Horseradish peroxidase (HRP) conjugated goat anti-cat immunoglobulins may be obtained from Jackson Immunological Co. or Sigma Chemical Company and others. For diagnosis of Bartonella, the conjugate is an anti-cat immunoglobulin having, in some embodiments, an enzyme chemically bound (conjugated) to it. In a preferred embodiment, horseradish peroxidase is coupled to the IgG fraction of anti-cat to confirm the presence or absence of antibodies 4 to antigen 3. In general, a conjugate at a certain dilution, such as 1:2K, is added to each well and incubated for a time and under certain conditions to allow at least a portion of conjugate 5 to bind to antibody 4, if present.

After incubation the serum and diluent are poured off and the wells washed sufficiently to remove serum remaining in any of wells 2. In preferred embodiments, plate 1 is washed at least 4 times with a buffer solution, such as PBS with Tween-20.

The stabilizing of the solution of step b) may be achieved by storing it in an HRP conjugate stabilizing solution at 4° C., which keeps the enzyme labeleds antibody stable and substantially pure. In a particular embodiment, the HRP conjugate stabilizing solution contains 50% volume/volume distilled water and glycerol.

In certain embodiments, a substrate will not have been reacted with the conjugate prior to binding the conjugate to the antibody. In such cases, a substrate, such as TMB substrate (commercially available from_Kirkgaard and Perry Laboratories) may be added, in a volume sufficient to coat, to wells 2. A substrate can be a chromogen, such as 3,3′,5,5′-tetramethylbenzidine, e.g. TMB (sold by _ Kirkgaard and Perry _ (see U.S. Pat. No. 5,013,646, which is hereby incorporated by reference) to allow visualization of bound antibody 4.

In various embodiments, Substrate 6 can be any one of a kind which react with conjugate 5. In certain embodiments, substrate 6 produces a colored component. For example, peroxidase such as that obtained from horseradish, produces a blue color when reacted with aminosalicylic acid and hydrogen peroxide, or p-phenylene diamine and hydrogen peroxide, or tetra-methyl benzidine and hydrogen peroxide. Other materials, like uric oxide, may be used to replace hydrogen peroxide as the acceptor. Alkaline phosphatase produces a yellow color when reacted with dinitrophenylphosphate. Beta galactosidase reacts with 0-nitrophenyl-beta-D-galactophyranoside to give a purple color.

Some common conjugates with enzyme labels useful in carrying out the method of this invention are horseradish peroxidase, alkaline phosphatase, glucoamylase, carbonic anhydrase, acetylcholinesterase, glucose oxidase, urease and beta-galactosidase. Other enzymes, such as those listed found in U.S. Pat. No. 4,275,149, are acceptable.

A variety of substrates and chromophores are available for use with these enzymes. Horseradish peroxidase, for instance, employs H₂O₂ and one or more of the following example chromogens to generate a colored product: 5-amino salicylic acid, 2,2′-azino-bis (3-ethylbenzthiazoline-6-sulfamic acid), o-dianisidine, o-phenylenediamine and 3,3′,5,5′-tetramethylbenzidine, and the like. Other examples for this and other enzymes are cited in U.S. Pat. No. 4,299,916, hereby incorporated by reference. Further examples of suitable chromophores are the peroxidases, which require a chromogenic substrate and an acceptor such as hydrogen peroxide or uric oxide, and the hydrolases, which require only a chromogenic substrate.

Methods of the present invention may further include a computerized reading protocol for the determination of the antibodies in the samples. An example of an acceptable reader is one similar to the type described by Trottier, Y. L. et al. (1992, J. Clin. Microbiol., 30:46-53). Other embodiments use dual wavelength readers, such as Elx808BioTek. However, readers are well known in the art and any suitable reader will suffice.

In various embodiments, a reader of the present invention is a portable reader. Portable readers allow the embodiments of the present invention to be taken into the field during operations. Other embodiments utilize a color chart that may be visually read, as is common in the art.

In accordance with another embodiment of the present invention there is provided an ELISA diagnostic kit for the assay of Bartonella antibodies in the serum of an organism, such as a cat comprising, in separate packaging, at least one of the following:

a) a plate or solid support having bound thereto a purified Bartonella antigen for a specific binding to anti-Bartonella antibodies present in the serum of cats;

b) serum from cats experimentally inoculated with a species, subspecies, and/or strain of Bartonella to serve as a positive control;

c) cat serum from a specific pathogen-free colony to serve as a negative control; and

d) an enzyme-labeled conjugate which binds to the cat antibodies bound to the plate or other solid phase.

The antigen of step a), when bound to a solid support, may be stabilized by storing it at 4° C. in the coating buffer. However, other stabilizing procedures may be utilized.

The ELISA diagnostic kits of the present invention may further comprise the following:

e) a substrate which allows the visualization of the detectably labeled conjugate.

In accordance with another embodiment of the present invention there is provided a method for the preparation of the kit, which comprises the steps of:

a) purifying Bartonella antigen by and centrifugation of said antigen bacterial crude extract; b) fixing the antigen of step a) to a solid support and stabilizing said fixed antigen; c) immunizing mammals with a strain of Bartonella and collecting serum to serve as positive control sera; and d) collecting sera from Bartonella-free colonies to serve as negative control sera.

Further embodiments of a diagnostic kit of the present invention include pre-packaged positive (not currently commercially available) and/or negative serum.

The kits of the present invention are novel in that they utilize a novel lysate and allow for a simple and fast testing in the field, such as a veterinary office or a research laboratory. The kits of the present invention are sufficiently stable in that they have a shelf life of about 3 months. The improved sensitivity and shelf life is a product of the novel lysate preparation of the present invention.

A kit of the present invention is easily used and provides rapid results. The kit can be used by a veterinarian having a minimum of experience, it may be used in the field where the animals are kept and does not require laboratory skills, since only simple steps need to be performed. In addition, this kit was demonstrated to give highly reliable and reproducible results.

Other accessories that may be included with kits of the present invention include spatulas, vials, deionized water, pre-mixed buffers, blocking agents, and/or the like. However, other test kit design may be apparent to those of skill in the art and all such kits are intended to be covered by the present invention.

The invention is further illustrated by the following examples:

### EXAMPLES

It is to be noted that the Examples include a number of microbiology and immunology techniques considered to be known to those of ordinary skill in the art. Disclosure of such techniques can be found, for example, in Prescott, et al., Microbiology, 3^{rd} Edition, Wm. C. Brown and Company; and Harlow, et al., 1988, Antibodies, a Laboratory Manual, Cold Spring Harbor Labs Press, which is hereby incorporated by reference. As well, the specific reagents and protocols for use in the detection methods described herein and similar indirect immunocytochemical methods can be selected from those available in the art based upon established criteria, such as that found in Antibodies: A Laboratory Manual, Harlow and Lane, Cold Spring Harbor Laboratory, Cold Spring Harbor, N.Y. 1988), the text of which is incorporated by reference. It will also be understood by those of ordinary skill in the art that these examples are merely illustrative and other procedures can be used. For an understanding of the scope of the patent, attention should be directed to the claims following the examples.

### Example 1

### Sample Definition

To compare the novel lysate assay of the present invention, 29 samples were run under the novel lysate preparation of the present invention and 29 samples were run under an Outer Membrane Protein (OMP) preparation of the prior art. The samples are defined as follows:

| Sample Number | Sample Description |
|---|---|
| 1 | Cat 1, prior to challenge |
| 2 | Cat 2, prior to challenge |
| 3 | Cat 3, prior to challenge |
| 4 | Cat 4, prior to challenge |
| 5 | Cat 5, prior to challenge |
| 6 | Cat 6, prior to challenge |
| 7 | Cat 1, 4 weeks post challenge |
| 8 | Cat 2, 4 weeks post challenge |
| 9 | Cat 3, 4 weeks post challenge |
| 10 | Cat 4, 4 weeks post challenge |
| 11 | Cat 5, 4 weeks post challenge |
| 12 | Cat 6, 4 weeks post challenge |
| 13 | Cat 1, 12 weeks post challenge |
| 14 | Cat 2, 12 weeks post challenge |
| 15 | Cat 3, 12 weeks post challenge |
| 16 | Cat 4, 12 weeks post challenge |
| 17 | Cat 5, 12 weeks post challenge |
| 18 | Cat 6, 12 weeks post challenge |
| 19 | Cat 7, challenged with Chlamydia |
| 20 | Cat 8, challenged with Chlamydia |
| 21 | Cat 9, challenged with Chlamydia |
| 22 | Cat 10, challenged with Chlamydia |
| 23 | Cat 11, challenged with Chlamydia |
| 24 | Cat 12, challenged with Chlamydia |
| 25 | Cat 13, challenged with Chlamydia |
| 26 | Cat 14, challenged with Chlamydia |
| 27 | Cat 15, challenged with Chlamydia |
| 28 | Negative B. henselae serum |
| 29 | Positive B. henselae serum |

### Example 2

### Growing Bartonella

a. For Triton Lysate

This Example describes the preparation and formulation of a Bartonella henselae antigen (Ags) for use in embodiments of the present invention.

In an experiment, ten 150 cm² flasks of Columbia blood agar were inoculated with 1.5 ml of a 1:6 dilution of B. henselae, although other Bartonella species could be used. The inoculated flasks were incubated in a Forma Scientific water-jacketed incubator at 37° C. in an atmosphere of 10% CO₂. Cultures were grown for 72 hours. Cultures were harvested with sterile 2 mm diameter glass beads and approximately 3 ml of 10 mM HEPES (Sigma) per flask. The dislodged bacteria were collected by pipette from the flasks. The glass beads were rinsed with 10 ml HEPES per flask to collect remaining dislodged bacteria.

The collected bacteria were then pooled in an Oakridge tube and centrifuged in a Mistral 3000 i centrifuge at 3,000 RPM for 10 minutes in an environment of 4° C. The pellet from the centrifugation was then resuspended in 25 ml of a borate saline solution at pH 9. The borate saline solution had a composition of 80 ml 1.5 M NaCl, 100 ml 0.5 M H₃BO₃, 24 ml 1.0 N NaOH, and 796 ml distilled H₂O. The pellet resuspended in the borate saline solution was then vortexed and centrifuged as above.

All material except the resulting pellet was discarded. The pellet was then resuspended in 6 ml of Borate Saline solution at pH 9 with 1% Triton X-100 (sold by Triton). The pellet was then sonicated in a Branson Sonifier Model 450 for 5 minutes at a 50% duty cycle, maximum power level with the probe of the sonicator inserted into the liquid portion. The sonication was performed over ice for temperature control. The sonicated portion was then centrifuged as above. The resulting supernatant was then aliquoted into 1 ml samples and froze at −20° C. until use.

b. for OMP

This portion of the example describes the preparation and formulation of a Bartonella henselae outer membrane protein for use in embodiments of the present invention:

1) B. henselae was grown on Columbia blood agar—5 175 cm² flasks.

2) B. henselae was harvested with sterile glass beads, 10 ml 10 mM HEPES per flask.

3) The cells were pelleted @3000 RPM in centrifuge (Mistral 3000 i) at 20° C. for 20 minutes; discard supernatant, store overnight at 4° C.

4) The pellet was resuspended in 10 ml 10 mm HEPES and pellet resuspension @3000 RPM in centrifuge (Mistral 3000 i) at 20° C. for 10 minutes; discard supernatant and resuspend pellet in 10 ml 10 mM HEPES. The suspended pellet was then placed on ice.

5) The suspended pellet was then sonicated with a Branson 450 Sonifier using a microtip submerged at 70% duty cycle, output=6 (6/10) for 15 minutes at 15 second intervals with 45 second rest intervals.

6) The sonicated pellet was then centrifuged in 1 ml aliquots at 13,000 RPM at 4° C. in an Eppendorf Centrifuge 5402 for 2 minutes. The supernatant was then transferred to a clean tube and centrifuged as above for 30 minutes.

7) Then, each pellet was resuspended in 200 ul 10 mM HEPES with 2% sarcosyl. Each resuspended pellet was then placed on ice for one hour with mixing intermittently.

8) The resuspended pellet was then centrifuged as above for 30 minutes. Then the supernatant was removed.

9) The pellets were resuspended in 200 ul 10 mM HEPES and frozen at −20C.

C. Preparation of Antisera—Positive Control

Suitable positive antisera control may be purchased from a supplier, as is common in the art.

D. Preparation of Antisera-Negative Control

Suitable negative antisera control may be purchased from a supplier, as is common in the art.

### Example 3

### Coating of Plates

A. Triton Lysate with Bartonella henselae

1. Determining Appropriate Dilution

Table 1 illustrates absorbance values from an experiment in which Greiner plates were coated overnight at 4° C. with B. henselae triton lysate as prepared under Example 2. A known positive (control) serum was cross-titrated against the B. henselae triton lysate for determining a proper coating dilution of the novel triton lysate. Table 2 illustrates absorbance values for a known negative (control) serum that was cross-titrated against antigen on another plate coated as prepared under Example 1 using Greiner plates coated overnight at 4° C. To coat the plate, the Antigen (Ag) was serially diluted in Columns 1-6, starting at 1:500, going two-fold across the plate. Columns 7-12 contained only PBS.

The plates were blocked for 1 hour at 37° C. with 200 μl per well with serum diluent consisting of PBS with 5% Carnation dry non-fat milk, 0.1% tween-20, and 0.01% thimerosal. The plates were then washed one time with wash buffer consisting of PBS with 0.1% tween-20 and 0.01% thimerosal. For the plate described in Table 1, the positive serum for B. henselae was serially diluted in serum diluent 2-fold down the plate beginning at 1:1 K. For the plate described in Table 2, the negative serum for B. henselae was serially diluted in serum diluent 2-fold down the plate beginning at 1:1K. The plates were then incubated for 60 minutes at 37° C. and washed 4 times with wash buffer. The horseradish peroxidase-labeled secondary antibody of goat anti-cat IgG diluted 1:2K in serum diluent was then added, and the plates were incubated for 45 minutes at 37° C. The plates were then washed 4 times and 2-component TMB substrate was added. The plates were then incubated for ten minutes at room temperature in the dark and the color development stopped with 100 μl per well of 2M H₂SO₄. The absorbance was read on a microtiter plate reader (ICN Titertek Multiscan Bichromatic) at dual wavelength the absorbance at the reference wavelength (540 nm) was subtracted from the absorbance at the primary wavelength (450 nm) for each well.

Table 1

Columns numbered 1 to 6 illustrate the absorbance values of the serial dilution of the lysate coating antigen. Columns 7 to 12 illustrate the absorbance values of buffer-only coating antigen control for establishing a baseline. The row values are serial dilutions for the absorbance values of the serial dilution of the positive serum.

| | 1 | 2 | 3 | 4 | 5 | 6 | 7 | 8 | 9 | 10 | 11 | 12 |
|---|---|---|---|---|---|---|---|---|---|---|---|---|
| | Lysate Dilution | | | | | | | | | | | |
| | 1:500 | 1.1 K | 1:2 K | 1 4 K | 1:8 K | 1:16 K | N/A | N/A | N/A | N/A | N/A | N/A |
| 1:1 K | 2.765 | 2.853 | 2 669 | 2 506 | 2.293 | 2 312 | 0 017 | 0 018 | 0 042 | 0.026 | 0.026 | 0 014 |
| 2 K | 2.760 | 2 616 | 2 559 | 2 452 | 2.221 | 1 906 | 0 014 | 0 024 | 0.008 | 0 009 | 0.008 | 0.008 |
| 4 K | 2.554 | 2 391 | 2.230 | 2.045 | 1.775 | 1.486 | 0.006 | 0 007 | 0 010 | 0 017 | 0.009 | 0.008 |
| 8 K | 2.311 | 2 128 | 1.966 | 1 655 | 1 406 | 1 063 | 0.017 | 0 031 | 0 004 | 0.006 | 0.003 | 0.004 |
| 16 K | 1.887 | 1 652 | 1 446 | 1.214 | 0.947 | 0.725 | 0.016 | 0 006 | 0 003 | 0 024 | 0.005 | 0.006 |
| 32 K | 1.299 | 1 086 | 0 945 | 0 698 | 0 559 | 0 373 | 0 004 | 0.005 | 0.003 | 0 009 | 0 003 | 0 004 |
| 64 K | 0.778 | 0 627 | 0 497 | 0.446 | 0.327 | 0.236 | 0 006 | 0 004 | 0 002 | 0 002 | 0.007 | 0.005 |
| 128 K | 0.459 | 0.358 | 0.310 | 0 242 | 0 181 | 0.104 | 0 005 | 0.003 | 0.035 | 0 002 | 0 003 | 0.010 |

Table 2

Columns numbered 1 to 6 illustrate the absorbance values of the serial dilution of the lysate coating antigen. Columns 7 to 12 illustrate the absorbance values of buffer-only coating antigen control for establishing a baseline. The row values are serial dilutions for the absorbance values of the serial dilution of the negative serum.

| | 1 | 2 | 3 | 4 | 5 | 6 | 7 | 8 | 9 | 10 | 11 | 12 |
|---|---|---|---|---|---|---|---|---|---|---|---|---|
| | Lysate Dilution | | | | | | | | | | | |
| | 1:500 | 1.1 K | 1 2 K | 1 4 K | 1:8 K | 1 16 K | N/A | N/A | N/A | N/A | N/A | N/A |
| 1:1 K | 0.018 | 0 010 | 0.014 | 0.005 | 0 099 | 0 008 | 0 004 | 0.213 | 0.005 | 0 013 | 0 101 | 0.004 |
| 2 K | 0.011 | 0.006 | 0.009 | 0 004 | 0 003 | 0.012 | 0 089 | 0 115 | 0 060 | 0.003 | 0.003 | 0.001 |
| 4 K | 0.005 | 0 017 | 0 011 | 0.096 | 0 003 | 0 013 | 0 003 | 0.034 | 0 024 | 0.005 | 0 054 | 0 002 |
| 8 K | 0.003 | 0 004 | 0 002 | 0.007 | 0 004 | 0.011 | 0.004 | 0 054 | 0.004 | 0.004 | 0 003 | 0.002 |
| 16 K | 0 005 | 0 005 | 0 005 | 0 007 | 0 009 | 0 011 | 0.012 | 0.005 | 0.004 | 0 002 | 0.009 | 0 003 |
| 32 K | 0.004 | 0.004 | 0.003 | 0 010 | 0 003 | 0.008 | 0.003 | 0 004 | 0.002 | 0 003 | 0 003 | 0 002 |
| 64 K | 0 003 | 0 002 | 0.003 | 0.021 | 0.009 | 0 007 | 0 003 | 0.006 | 0.002 | 0.003 | 0 003 | 0.002 |
| 128 K | 0.003 | 0.006 | 0 008 | 0 003 | 0.004 | 0.007 | 0.002 | 0 031 | 0 017 | 0.003 | 0.003 | 0 009 |

The results illustrate that a lysate preparation of the present invention produces a very clean assay with nearly nil background. The optimal concentration for the coating appears to be between 1K and 2K.

2. Screening from Cats Infected with either B. henselae or Chlamydia

Six Greiner Microlon plates were coated with B. henselae triton lysate as prepared under Example 1 at a concentration of 1:2K and incubated overnight at 4° C. (value determined from previous plate) in PBS at pH 7.4 on the left side of the plate (+). The right side of the plate was coated with PBS only (−). The plates were washed 1 time, blocked for 45 minutes at 37° C. with 200 μl per well of serum diluent comprising PBS with 0.1% Tween-20, 0.01% thimerosal, and 5% Carnation dry non-fat milk. The plates were then washed 1 time and then serially diluted on both + and − side of plate starting at 1:1 K and proceeding 2 fold down plate. The plates were then incubated for 1 hour at 37 C, and washed 4 times. Then, 100 μl per well of Goat anti-cat IgG-HRP in a dilution of 1:2K in serum diluent (as defined above) was incubated on plates for 45 minutes at 37° C. The plates were then washed 4 times and 100 μl of K⁺ P TMB substrate was added per well. The plates were allowed to stand for ten minutes at room temperature in the dark and then stopped with 100 μl 2M H₂SO₄ and read immediately at 450 to 540 nm, as above.

Table 1

The raw absorbance values of the lysate preparation samples are presented below for six cats challenged with live B. henselae (1-6) prior to a first bleeding. The column headings are sera definitions as follows: Samples 1-6 are absorbance values from sera from challenged cats 1-6. Samples 1a-6a are absorbance values from buffer only columns used as controls for establishing a baseline. The row headings are dilutions. The definitions refer to the sample definitions given in Example 1.

| | Sample | | | | | | | | | | | |
|---|---|---|---|---|---|---|---|---|---|---|---|---|
| | 1 | 2 | 3 | 4 | 5 | 6 | 1a | 2a | 3a | 4a | 5a | 6a |
| | Definition | | | | | | | | | | | |
| | Cat 1 | Cat 2 | Cat 3 | Cat 4 | Cat 5 | Cat 6 | Control | Control | Control | Control | Control | Control |
| 1:1 K | 0.023 | 0.017 | 0.019 | 0.022 | 0 015 | 0 034 | 0 022 | 0 003 | 0 003 | 0.006 | 0.032 | 0.003 |
| 2 K | 0.006 | 0 008 | 0 015 | 0 020 | 0.042 | 0.019 | 0 008 | 0.003 | 0.001 | 0 003 | 0 003 | 0.004 |
| 4 K | 0.008 | 0 015 | 0 045 | 0.022 | 0 003 | 0 013 | 0 001 | 0 001 | 0 003 | 0 004 | 0.013 | 0 002 |
| 8 K | 0.003 | 0.006 | 0.002 | 0.038 | 0 024 | 0 017 | 0 003 | 0 007 | 0 003 | 0 039 | 0 014 | 0.012 |
| 16 K | 0.013 | 0 006 | 0 010 | 0 006 | 0.008 | 0 013 | 0 005 | 0 018 | 0.004 | 0.005 | 0 010 | 0 003 |
| 32 K | 0.006 | 0.001 | 0.007 | 0.009 | 0 002 | 0 006 | 0 002 | 0 007 | 0 000 | 0 040 | 0.052 | 0.002 |
| 64 K | 0.005 | 0.005 | 0.001 | 0 009 | 0 006 | 0.023 | 0.003 | 0 058 | 0.001 | 0.004 | 0.002 | 0 005 |
| 128 K | 0.026 | 0.003 | 0.009 | 0.031 | 0.044 | 0 010 | 0.002 | 0.034 | 0 062 | 0 006 | 0.002 | 0.029 |

Table 2

The raw absorbance values of the lysate preparation are presented below for the six cats above at four weeks post-challenge. The column headings are sera definitions as follows: Samples 7-12 are absorbance values from sera from challenged cats 1-6 four weeks post-challenge. Samples 7a-12a are absorbance values from buffer-only columns used as controls for establishing a baseline. The row headings are serum dilutions. The definitions refer to the sample definitions given in Example 1.

| | Samples | | | | | | | | | | | |
|---|---|---|---|---|---|---|---|---|---|---|---|---|
| | 7 | 8 | 9 | 10 | 11 | 12 | 7a | 8a | 9a | 10a | 11a | 12a |
| | Definitions | | | | | | | | | | | |
| | Cat 1 | Cat 2 | Cat 3 | Cat 4 | Cat 5 | Cat 6 | Control | Control | Control | Control | Control | Control |
| 1:1 K | 1.64 | 0 021 | 0.051 | 1 525 | 0 283 | 1 957 | 0.007 | 0.004 | 0 013 | 0.01 | 0 011 | 0.003 |
| 2 K | 0.926 | 0.042 | 0 016 | 1.176 | 0 113 | 1 435 | 0 004 | 0 016 | 0 029 | 0 004 | 0.003 | 0.003 |
| 4 K | 0.739 | 0.005 | 0.016 | 0 696 | 0 071 | 0 973 | 0.001 | 0.003 | 0 009 | 0 014 | 0.006 | 0.001 |
| 8 K | 0.422 | 0 005 | 0 004 | 0.415 | 0.031 | 0598 | 0 003 | 0 015 | 0.006 | 0 002 | 0.002 | 0 002 |
| 16 K | 0 228 | 0.007 | 0.012 | 0 245 | 0.052 | 0 315 | 0.014 | 0.004 | 0 002 | 0 011 | 0.003 | 0.003 |
| 32 K | 0.115 | 0 005 | 0 006 | 0.126 | 0 007 | 0.179 | 0 001 | 0 006 | 0.001 | 0 000 | 0.002 | 0.001 |
| 64 K | 0.056 | 0.004 | 0 004 | 0 076 | 0.021 | 0 091 | 0.002 | 0.002 | 0 001 | 0.001 | 0 002 | 0.003 |
| 128 K | 0.029 | 0 008 | 0.002 | 0.03 | 0 005 | 0 056 | 0.000 | 0 009 | 0 044 | 0 025 | 0 02 | 0 012 |

Table 3

The raw absorbance values of the lysate preparation are presented below for the six cats above twelve weeks post-challenge. The column headings are sera definitions as follows: Samples 13-18 are absorbance values from sera from challenged cats 1-6 twelve weeks post-challenge. Samples 13a-18a are absorbance values from buffer-only columns used as controls for establishing a baseline. The row headings are dilutions. The definitions refer to the sample definitions given in Example 1.

| | Sample | | | | | | | | | | | |
|---|---|---|---|---|---|---|---|---|---|---|---|---|
| | 13 | 14 | 15 | 16 | 17 | 18 | 13a | 14a | 15a | 16a | 17a | 18a |
| | Definition | | | | | | | | | | | |
| | Cat 1 | Cat 2 | Cat 3 | Cat 4 | Cat 5 | Cat 6 | Control | Control | Control | Control | Control | Control |
| 1:1 K | 2.144 | 0.035 | 0.093 | 2.261 | 0 237 | 2 727 | 0.010 | 0.030 | 0.023 | 0.014 | 0 017 | 0.003 |
| 2 K | 1.440 | 0 013 | 0 056 | 1 765 | 0 111 | 2 275 | 0 007 | 0 003 | 0.013 | 0 003 | 0.043 | 0 001 |
| 4 K | 0.977 | 0.008 | 0 031 | 1 125 | 0 060 | 1 728 | 0.003 | 0 002 | 0 005 | 0.013 | 0 005 | 0.003 |
| 8 K | 0.544 | 0.007 | 0 015 | 0.640 | 0 033 | 1.081 | 0.005 | 0 003 | 0 004 | 0 002 | 0 002 | 0 003 |
| 16 K | 0.302 | 0 008 | 0.020 | 0 334 | 0.027 | 0 652 | 0.009 | 0 004 | 0 002 | 0 003 | 0 014 | 0.056 |
| 32 K | 0.161 | 0 005 | 0 017 | 0.167 | 0.008 | 0.353 | 0.008 | 0 004 | 0.003 | 0 002 | 0 002 | 0 003 |
| 64 K | 0 076 | 0 001 | 0.003 | 0 085 | 0.006 | 0 181 | 0 005 | 0.005 | 0 003 | 0.002 | 0 006 | 0.003 |
| 128 K | 0.039 | 0 002 | 0 053 | 0.071 | 0 004 | 0.084 | 0 004 | 0 003 | 0.028 | 0 003 | 0.004 | 0 013 |

Table 4

The raw absorbance values of the lysate preparation are presented below for six cats challenged with Chlamydia. The column headings are sera definitions as follows: Samples 19-24 are absorbance values from sera from challenged cats. Samples 19a-24a are absorbance values from buffer only columns used as controls for establishing a baseline. The row headings are dilutions. The definitions refer to the sample definitions given in Example 1.

| | Samples | | | | | | | | | | | |
|---|---|---|---|---|---|---|---|---|---|---|---|---|
| | 19 | 20 | 21 | 22 | 23 | 24 | 19a | 20a | 21a | 22a | 23a | 24a |
| | Definitions | | | | | | | | | | | |
| | Cat 7 | Cat 8 | Cat 9 | Cat 10 | Cat 11 | Cat 12 | Control | Control | Control | Control | Control | Control |
| 1:1 K | 0.013 | 0.025 | 0.081 | 0 011 | 0 018 | 0.033 | 0.004 | 0.021 | 0 006 | 0 011 | 0.081 | 0.003 |
| 2 K | 0.006 | 0 011 | 0.018 | 0 011 | 0.062 | 0.017 | 0.003 | 0 094 | 0 002 | 0.004 | 0.004 | 0.003 |
| 4 K | 0.007 | 0.008 | 0 011 | 0.007 | 0.007 | 0 032 | 0 002 | 0 005 | 0 002 | 0.010 | 0.006 | 0.004 |
| 8 K | 0.007 | 0.010 | 0.004 | 0.026 | 0 003 | 0.011 | 0.006 | 0.008 | 0.003 | 0 002 | 0 001 | 0 004 |
| 16 K | 0 003 | 0 013 | 0.017 | 0.009 | 0 009 | 0.013 | 0.008 | 0.003 | 0 002 | 0.002 | 0.005 | 0 005 |
| 32 K | 0.003 | 0 009 | 0.002 | 0.015 | 0.002 | 0 011 | 0 007 | 0 002 | 0 005 | 0 010 | 0.002 | 0 003 |
| 64 K | 0.005 | 0 004 | 0 002 | 0.021 | 0 009 | 0 029 | 0 003 | 0.008 | 0 002 | 0.001 | 0.016 | 0 016 |
| 128 K | 0 003 | 0 015 | 0.013 | 0.003 | 0 002 | 0 007 | 0 004 | 0 006 | 0.054 | 0.003 | 0 006 | 0.084 |

Table 5

The raw absorbance values of the lysate preparation are presented below for the three cats (13-15) challenged with Chlamydia. Further, a positive serum sample and a negative serum sample were run. The column headings are sera definitions as follows: Samples 25-27 are absorbance values from sera from challenged cats 13-15. Sample 28 is a negative serum control. Sample 29 is a positive serum control. Samples 30-34 are absorbance values from buffer-only columns used as controls for establishing a baseline. The row headings are dilutions. The definitions refer to the sample definitions given in Example 1.

| | Samples | | | | | | | | | |
|---|---|---|---|---|---|---|---|---|---|---|
| | 25 | 26 | 27 | 28 | 29 | 30 | 31 | 32 | 33 | 34 |
| | Definitions | | | | | | | | | |
| | Cat 13 | Cat 14 | Cat 15 | Ser.− | Ser.+ | Control | Control | Control | Control | Control |
| 1:1 K | 0 017 | 0.007 | 0.044 | 0 027 | 2 932 | 0.004 | 0.005 | 0.007 | 0 007 | 0 04 |
| 2 K | 0 007 | 0 013 | 0 014 | 0 022 | 2.948 | 0 008 | 0 003 | 0 004 | 0.011 | 0 018 |
| 4 K | 0.068 | 0.012 | 0 007 | 0 009 | 2 720 | 0.005 | 0.004 | 0.009 | 0 018 | 0 039 |
| 8 K | 0 009 | 0 013 | 0 009 | 0.041 | 2 185 | 0 008 | 0 008 | 0.009 | 0.003 | 0 010 |
| 16 K | 0 004 | 0 017 | 0.025 | 0.013 | 1 672 | 0.009 | 0 024 | 0 019 | 0 008 | 0.008 |
| 32 K | 0 002 | 0 003 | 0 005 | 0 024 | 1.143 | 0 006 | 0 028 | 0 006 | 0.003 | 0 005 |
| 64 K | 0.011 | 0 009 | 0.011 | 0.031 | 0 687 | 0.016 | 0 017 | 0 003 | 0 019 | 0.011 |
| 128 K | 0.002 | 0 009 | 0 005 | 0 004 | 0.381 | 0 031 | 0 002 | 0.010 | 0.004 | 0 001 |

B. OMP

For comparison, an assay employing a B. henselae Outer Membrane Protein (OMP) antigen (Ag) preparation as the coating antigen was run according to the procedure for the novel lysate above.

Six Greiner Microlon plates were coated overnight at 4° C. with B. henselae OMP as prepared under Example 2 at a concentration of 187 ng/ml in 0.05 M carbonate buffer at pH 9.6 on the left side of the plate. The right side of the plates was coated with 0.05 M carbonate buffer only. The plates were washed once, blocked with 200 μl per well serum diluent and incubated one hour at 37° C. The plates were then washed once, and serum serially diluted on both + and − side of plate starting at 1:1K and proceeding 2-fold down plate. The plates were incubated for 1 hour at 37° C., then washed 4 times. Next, 100 μl per well of Goat anti-cat IgG-HRP at a dilution of 1:2K in serum diluent (as defined above) was incubated on plates for 45 minutes at 37° C. The plates were then washed 4 times and 100 μl of K⁺ P TMB substrate was added to each well. The plates were incubated for ten minutes in the dark at room temperature and then stopped with 100 μl per well 2M H₂SO₄ and read immediately at 450 minus 540 nm, as above.

Table 1

The raw absorbance values of the OMP preparation are presented below for the challenged cats above prior to a first bleeding and correspond to the sera for the lysate preparation taken prior to the first bleeding. The column headings are samples as follows: Samples 1-6 are absorbance values from sera from challenged cats 1-6. Samples 1b-6b are absorbance values from buffer-only columns used as controls. The row headings are dilutions. The definitions refer to the sample definitions given in Example 1.

| | Samples | | | | | | | | | | | |
|---|---|---|---|---|---|---|---|---|---|---|---|---|
| | 1 | 2 | 3 | 4 | 5 | 6 | 1b | 2b | 3b | 4b | 5b | 6b |
| | Definition | | | | | | | | | | | |
| | Cat 1 | Cat 2 | Cat 3 | Cat 4 | Cat 5 | Cat 6 | Control | Control | Control | Control | Control | Control |
| 1:1 K | 0.270 | 0.118 | 0 121 | 0 200 | 0.172 | 0 203 | 0 012 | 0.081 | 0.010 | 0.021 | 0.013 | 0 008 |
| 2 K | 0.148 | 0 076 | 0.085 | 0.140 | 0 145 | 0.121 | 0.011 | 0.056 | 0 008 | 0 009 | 0 008 | 0 008 |
| 4 K | 0.090 | 0.047 | 0 147 | 0 076 | 0.112 | 0.162 | 0.027 | 0 049 | 0.008 | 0 014 | 0 011 | 0.009 |
| 8 K | 0 042 | 0 028 | 0.025 | 0.054 | 0.030 | 0 049 | 0 029 | 0.036 | 0.026 | 0.009 | 0.130 | 0 008 |
| 16 K | 0.023 | 0.019 | 0 029 | 0 028 | 0 025 | 0.034 | 0.033 | 0 012 | 0 008 | 0 009 | 0 015 | 0.009 |
| 32 K | 0.017 | 0.018 | 0.013 | 0.023 | 0 017 | 0 082 | 0.012 | 0 012 | 0.008 | 0.009 | 0.009 | 0.009 |
| 64 K | 0 252 | 0 019 | 0 012 | 0 104 | 0.015 | 0 026 | 0.010 | 0 026 | 0 008 | 0 008 | 0 075 | 0.015 |
| 128 K | 0.017 | 0 010 | 0.010 | 0.012 | 0.012 | 0 142 | 0 010 | 0 010 | 0 010 | 0 009 | 0.009 | 0.018 |

Table 2

The raw absorbance values of the OMP preparation are presented below for the six cats above four weeks post-challenge and correspond to the sera for the lysate preparation taken four weeks post-challenge. The column headings are samples as follows: Samples 7-12 are absorbance values for sera from the challenged cats four weeks post-challenge. Samples 7b-12b are absorbance values from buffer-only columns used as controls. The row headings are dilutions. The definitions refer to the sample definitions given in Example 1.

| | Sample | | | | | | | | | | | |
|---|---|---|---|---|---|---|---|---|---|---|---|---|
| | 7 | 8 | 9 | 10 | 11 | 12 | 7b | 8b | 9b | 10b | 11b | 12b |
| | Definition | | | | | | | | | | | |
| | Cat 1 | Cat 2 | Cat 3 | Cat 4 | Cat 5 | Cat 6 | Control | Control | Control | Control | Control | Control |
| 1:1 K | 2.290 | 0 126 | 0 441 | 2 156 | 0.208 | 2.382 | 0 012 | 0 139 | 0 010 | 0.017 | 0 015 | 0.010 |
| 2 K | 1.792 | 0.070 | 0 294 | 1 909 | 0 195 | 2.610 | 0 061 | 0 010 | 0 010 | 0.012 | 0 009 | 0 009 |
| 4 K | 1 525 | 0.051 | 0.223 | 1.592 | 0.118 | 1 829 | 0 008 | 0 046 | 0 009 | 0 021 | 0.083 | 0.008 |
| 8 K | 0 987 | 0 039 | 0 082 | 1 305 | 0 053 | 1.522 | 0.015 | 0 010 | 0.009 | 0.013 | 0 009 | 0 009 |
| 16 K | 0.652 | 0.025 | 0.052 | 0.864 | 0.033 | 1 174 | 0 038 | 0 010 | 0 010 | 0 035 | 0.013 | 0 010 |
| 32 K | 0 323 | 0 018 | 0 024 | 0 520 | 0.017 | 0.732 | 0 012 | 0 009 | 0.009 | 0 008 | 0 008 | 0.010 |
| 64 K | 0.156 | 0 012 | 0 014 | 0 284 | 0.017 | 0.432 | 0 010 | 0 053 | 0 060 | 0.008 | 0.012 | 0 012 |
| 128 K | 0 090 | 0 011 | 0 138 | 0.138 | 0.011 | 0.208 | 0 016 | 0 010 | 0 037 | 0 008 | 0.009 | 0.025 |

Table 3

The raw absorbance values of the OMP preparation are presented below for the six cats (1-6) above twelve weeks post-challenge and correspond to the sera for the lysate preparation taken twelve weeks post-challenge. The column headings are samples as follows: Samples 13-18 are absorbance values from sera from the challenged cats twelve weeks post-challenge. Samples 13b-18b are absorbance values from buffer-only columns used as controls for establishing a baseline. The row headings are dilutions. The definitions refer to the sample definitions given in Example 1.

| | Samples | | | | | | | | | | | |
|---|---|---|---|---|---|---|---|---|---|---|---|---|
| | 13 | 14 | 15 | 16 | 17 | 18 | 13b | 14b | 15b | 16b | 17b | 18b |
| | Definition | | | | | | | | | | | |
| | Cat 1 | Cat 2 | Cat 3 | Cat 4 | Cat 5 | Cat 6 | Control | Control | Control | Control | Control | Control |
| 1:1 K | 1.945 | 0 115 | 0.188 | 2 177 | 0.210 | 2 285 | 0.010 | 0.014 | 0.025 | 0.009 | 0.009 | 0 009 |
| 2 K | 1.540 | 0 071 | 0.100 | 1.641 | 0.297 | 2 520 | 0 012 | 0.009 | 0.008 | 0.009 | 0.008 | 0 013 |
| 4 K | 1.113 | 0.045 | 0.057 | 1.158 | 0.130 | 1 726 | 0 009 | 0.010 | 0.007 | 0.014 | 0.010 | 0.008 |
| 8 K | 0.682 | 0.029 | 0.027 | 0.677 | 0.070 | 1 380 | 0 011 | 0.010 | 0.007 | 0.009 | 0.009 | 0.009 |
| 16 K | 0.364 | 0.016 | 0.035 | 0.428 | 0.060 | 0.987 | 0 019 | 0.010 | 0 008 | 0.010 | 0.010 | 0.009 |
| 32 K | 0.176 | 0.013 | 0.013 | 0.220 | 0.021 | 0.650 | 0.010 | 0.010 | 0.009 | 0.009 | 0.009 | 0 009 |
| 64 K | 0 089 | 0.010 | 0 011 | 0.119 | 0 019 | 0 394 | 0 012 | 0.008 | 0 008 | 0 009 | 0 026 | 0 010 |
| 128 K | 0 049 | 0.008 | 0 009 | 0 059 | 0 013 | 0 198 | 0 009 | 0 007 | 0 031 | 0 090 | 0 008 | 0 017 |

Table 4

The raw absorbance values of the OMP preparation are presented below for six cats (7-12) challenged with Chlamydia and correspond to the sera for the lysate preparation taken above for cats 7-12. The column headings are samples as follows: Samples 19-24 are absorbance values from sera from challenged cats 7-12. Samples 19b-24b are absorbance values from buffer-only columns used as controls for establishing a baseline. The row headings are dilutions. The definitions refer to the sample definitions given in Example 1.

| | Samples | | | | | | | | | | | |
|---|---|---|---|---|---|---|---|---|---|---|---|---|
| | 19 | 20 | 21 | 22 | 23 | 24 | 19b | 20b | 21b | 22b | 23b | 24b |
| | Definition | | | | | | | | | | | |
| | Cat 7 | Cat 8 | Cat 9 | Cat 10 | Cat 11 | Cat 12 | Control | Control | Control | Control | Control | Control |
| 1:1 K | 0.043 | 0.112 | 0.063 | 0.034 | 0.067 | 0 017 | 0 010 | 0 010 | 0.010 | 0 025 | 0 021 | 0 009 |
| 2 K | 0 018 | 0 055 | 0 040 | 0 018 | 0 035 | 0.017 | 0 008 | 0 009 | 0 007 | 0 010 | 0.017 | 0.008 |
| 4 K | 0.027 | 0.045 | 0.024 | 0.012 | 0 025 | 0 020 | 0 009 | 0 008 | 0 008 | 0 017 | 0.016 | 0 009 |
| 8 K | 0 011 | 0 026 | 0.014 | 0 016 | 0.015 | 0 015 | 0 019 | 0 009 | 0 009 | 0.008 | 0.009 | 0.008 |
| 16 K | 0.007 | 0 019 | 0.029 | 0.013 | 0.030 | 0 026 | 0.028 | 0 011 | 0 014 | 0 008 | 0 029 | 0.009 |
| 32 K | 0 008 | 0 013 | 0 009 | 0 016 | 0 009 | 0 013 | 0 011 | 0 008 | 0 008 | 0 008 | 0.007 | 0 008 |
| 64 K | 0 007 | 0 010 | 0 008 | 0.020 | 0.010 | 0 014 | 0 013 | 0 008 | 0 007 | 0 008 | 0.016 | 0.011 |
| 128 K | 0 009 | 0 011 | 0 011 | 0 010 | 0 009 | 0 010 | 0 010 | 0.011 | 0 036 | 0.009 | 0 009 | 0 015 |

Table 5

The raw absorbance values of the OMP preparation are presented below for the three cats (13-15) challenged with Chlamydia and correspond to the sera for the lysate preparation taken above for cats 13-15. Further, a corresponding positive serum sample and negative serum sample were run. The column headings are samples as follows: Samples 25-27 are absorbance values from sera from challenged cats 13-15. Sample 28 is a negative serum control. Sample 29 is a positive serum control. Samples 25b-29b are absorbance values from buffer-only columns used as controls for establishing a baseline. The row headings are dilutions. The definitions are the definitions given in Example 1.

| | Samples | | | | | | | | | |
|---|---|---|---|---|---|---|---|---|---|---|
| | 25 | 26 | 27 | 28 | 29 | 25b | 26b | 27b | 28b | 29b |
| | Definition | | | | | | | | | |
| | Cat 13 | Cat 14 | Cat 15 | Ser− | Ser.+ | Control | Control | Control | Control | Control |
| 1:1 K | 0.023 | 0 092 | 0 128 | 0 100 | 2.792 | 0 023 | 0.011 | 0 009 | 0.018 | 0.026 |
| 2 K | 0 02 | 0 057 | 0.058 | 0.066 | 2 723 | 0.009 | 0.015 | 0 009 | 0 008 | 0 013 |
| 4 K | 0 014 | 0.031 | 0.029 | 0.059 | 2.481 | 0 008 | 0 026 | 0 008 | 0.013 | 0 018 |
| 8 K | 0.013 | 0.03 | 0 015 | 0 051 | 2.179 | 0 011 | 0 013 | 0.012 | 0 008 | 0 012 |
| 16 K | 0 009 | 0.018 | 0.021 | 0 02 | 1.728 | 0 018 | 0 011 | 0 008 | 0 008 | 0 011 |
| 32 K | 0.009 | 0.039 | 0 01 | 0 019 | 1 265 | 0 014 | 0 013 | 0.007 | 0.008 | 0.008 |
| 64 K | 0.008 | 0 009 | 0 014 | 0 021 | 0 730 | 0.021 | 0 008 | 0.007 | 0.016 | 0.019 |
| 128 K | 0.016 | 0.01 | 0 01 | 0 014 | 0 444 | 0 012 | 0 01 | 0.014 | 0.008 | 0.033 |

### Example 4

### Adjusted Raw Data Values

A. Lysate Preparation Adjusted Values

The values in the table below are the adjusted optical density values for the novel lysate preparation of the present invention. The adjusted values were computed by subtracting the raw OD value of the buffer-only (right) side of the plate from the corresponding well on the Ag-coated (left) side of the plate.

The cut-off optical density values were calculated by taking the mean of the buffer-only (right) side plus three (3) standard deviations of the buffer-only (right) side of the plate. The cut-off value for samples 1-6 was 0.058. The cut-off value for samples 7-12 was 0.032. The cut-off value for samples 13-18 was 0.040. The cut-off value for samples 19-24 was 0.074. The cut-off value for samples 25-29 was 0.037.

TABLE 1

| | Dilution | | | | | | | |
|---|---|---|---|---|---|---|---|---|
| | 1:1 K | 2 K | 4 K | 8 K | 16 K | 32 K | 64 K | 128 K |
| SX 1 | 0 00 | 0.00 | 0 01 | 0.00 | 0.01 | 0 00 | 0 00 | 0.02 |
| SX 2 | 0.01 | 0 01 | 0.010 | 0 00 | −0 01 | −0.01 | −0.05 | −0.03 |
| SX 3 | 0 02 | 0.04 | 0.04 | 0.00 | 0 01 | 0 01 | 0.00 | −0 05 |
| SX 4 | 0.02 | 0.02 | 0.02 | 0 00 | 0 00 | −0 03 | 0.01 | 0.03 |
| SX 5 | −0 02 | 0 04 | −0.01 | 0.01 | 0.00 | −0 05 | 0.00 | 0 04 |
| SX 6 | 0 03 | 0 02 | 0 01 | 0 01 | 0.01 | 0.00 | 0 02 | −0 02 |
| SX 7 | 1.63 | 0 92 | 0 74 | 0.42 | 0 21 | 0 11 | 0 05 | 0.03 |
| SX 8 | 0 02 | 0.03 | 0 00 | −0 01 | 0 00 | 0 00 | 0 00 | 0.00 |
| SX 9 | 0 04 | −0.01 | 0.01 | 0 00 | 0 01 | 0 01 | 0.00 | −0.04 |
| SX 10 | 1.52 | 1 17 | 0 68 | 0 41 | 0.23 | 0 13 | 0 08 | 0 00 |
| SX 11 | 0 27 | 0 11 | 0.06 | 0 03 | 0 05 | 0 01 | 0.02 | −0.02 |
| SX 12 | 1.95 | 1.43 | 0.97 | 0 60 | 0.31 | 0.18 | 0 09 | 0 04 |
| SX 13 | 2.13 | 1.43 | 0.97 | 0.54 | 0 29 | 0.15 | 0.07 | 0.04 |
| SX 14 | 0.01 | 0 01 | 0.01 | 0 00 | 0.00 | 0 00 | 0 00 | 0 00 |
| SX 15 | 0 07 | 0.04 | 0.03 | 0 01 | 0 02 | 0.01 | 0.00 | 0.03 |
| SX 16 | 2.25 | 1.76 | 1.11 | 0 64 | 0.33 | 0 17 | 0 08 | 0.07 |
| SX 17 | 0 22 | 0 07 | 0.06 | 0.03 | 0 02 | 0 01 | 0.00 | 0 00 |
| SX 18 | 2 72 | 2 27 | 1 73 | 1 08 | 0.60 | 0 35 | 0 18 | 0 07 |
| SX 19 | 0 01 | 0 00 | 0.01 | 0 00 | −0 01 | 0 00 | 0.00 | 0.00 |
| SX 20 | 0 00 | −0 08 | 0 00 | 0.00 | 0 01 | 0 01 | 0 00 | 0.01 |
| SX 21 | 0 08 | 0 02 | 0.01 | 0 00 | 0 02 | 0 00 | 0.00 | −0.04 |
| SX 22 | 0 00 | 0 01 | 0 00 | 0 02 | 0.01 | 0 01 | 0 02 | 0.00 |
| SX 23 | −0 06 | 0 06 | 0.00 | 0.00 | 0 00 | 0.00 | −0.01 | 0.00 |
| SX 24 | 0 03 | 0.01 | 0 03 | 0 01 | 0.01 | 0 01 | 0 01 | −0 08 |
| SX 25 | 0.01 | 0 00 | 0 06 | 0.00 | −0 01 | 0 00 | −0 01 | −0.03 |
| SX 26 | 0 00 | 0.01 | 0 01 | 0 01 | −0 01 | −0.03 | −0 01 | 0.01 |
| SX 27 | 0.04 | 0.01 | 0 00 | 0.00 | 0 01 | 0 00 | 0 01 | −0.01 |
| SX 28 | 0.02 | 0 01 | −0.01 | 0 04 | 0 01 | 0.02 | 0.01 | 0.00 |
| SX 29 | 2.89 | 2.93 | 2 68 | 2 18 | 1.66 | 1 14 | 0 68 | 0 38 |

The assay was very clean. There was little, if any, background OD signal on the right side of the plate.

B. OMP Preparation Adjusted Values

The values in the table below are the adjusted optical density values for the prior art OMP preparation of the present invention. The adjusted values were computed by subtracting the raw OD value of the buffer-only (right) side of the plate from the corresponding well on the Ag-coated (left) side of the plate.

The cut-off optical density values were calculated by taking the mean of the buffer only (right) side plus three (3) standard deviations of the buffer-only (right) side of the plate. The cut-off value for samples 1-6 was 0.088. The cut-off value for samples 7-12 was 0.092. The cut-off value for samples 13-18 was 0.049. The cut-off value for samples 19-24 was 0.030. The cut-off value for samples 25-29 was 0.029.

TABLE 2

| | Dilution | | | | | | | |
|---|---|---|---|---|---|---|---|---|
| | 1:1 K | 2 K | 4 K | 8 K | 16 K | 32 K | 64 K | 128 K |
| SX 1 | 0.26 | 0.14 | 0.06 | 0.01 | −0.01 | 0.01 | 0.24 | 0.01 |
| SX 2 | 0.04 | 0.02 | 0.00 | −0.01 | 0.01 | 0.01 | −0.01 | 0.00 |
| SX 3 | 0.11 | 0.08 | 0.14 | 0.00 | 0.02 | 0.01 | 0.00 | 0.00 |
| SX 4 | 0.18 | 0.13 | 0.06 | 0.05 | 0.02 | 0.01 | 0.10 | 0.00 |
| SX 5 | 0.16 | 0.14 | 0.10 | −0.10 | 0.01 | 0.01 | −0.06 | 0.00 |
| SX 6 | 0.20 | 0.11 | 0.15 | 0.04 | 0.03 | 0.07 | 0.01 | 0.12 |
| SX 7 | 2.28 | 1.73 | 1.52 | 0.97 | 0.61 | 0.31 | 0.15 | 0.07 |
| SX 8 | −0.01 | 0.06 | 0.00 | 0.03 | 0.02 | 0.01 | −0.04 | 0.00 |
| SX 9 | 0.43 | 0.28 | 0.21 | 0.07 | 0.04 | 0.02 | −0.05 | 0.10 |
| SX 10 | 2.14 | 1.90 | 1.57 | 1.29 | 0.83 | 0.51 | 0.28 | 0.13 |
| SX 11 | 0.19 | 0.19 | 0.03 | 0.04 | 0.02 | 0.01 | 0.01 | 0.00 |
| SX 12 | 2.37 | 2.60 | 1.82 | 1.51 | 1.16 | 0.72 | 0.42 | 0.18 |
| SX 13 | 1.94 | 1.53 | 1.10 | 0.67 | 0.35 | 0.17 | 0.08 | 0.04 |
| SX 14 | 0.10 | 0.06 | 0.04 | 0.02 | 0.01 | 0.00 | 0.00 | 0.00 |
| SX 15 | 0.16 | 0.09 | 0.05 | 0.02 | 0.03 | 0.00 | 0.00 | −0.02 |
| SX 16 | 2.17 | 1.63 | 1.14 | 0.67 | 0.42 | 0.21 | 0.11 | −0.03 |
| SX 17 | 0.20 | 0.29 | 0.12 | 0.06 | 0.05 | 0.01 | −0.01 | 0.01 |
| SX 18 | 2.28 | 2.51 | 1.72 | 1.37 | 0.98 | 0.64 | 0.38 | 0.18 |
| SX 19 | 0.03 | 0.01 | 0.02 | −0.01 | −0.02 | 0.00 | −0.01 | 0.00 |
| SX 20 | 0.10 | 0.05 | 0.04 | 0.02 | 0.01 | 0.01 | 0.00 | 0.00 |
| SX 21 | 0.05 | 0.03 | 0.02 | 0.01 | 0.02 | 0.00 | 0.00 | −0.03 |
| SX 22 | 0.01 | 0.01 | −0.01 | 0.01 | 0.01 | 0.01 | 0.01 | 0.00 |
| SX 23 | 0.05 | 0.02 | 0.01 | 0.01 | 0.00 | 0.00 | −0.01 | 0.00 |
| SX 24 | 0.01 | 0.01 | 0.01 | 0.01 | 0.02 | 0.01 | 0.00 | −0.01 |
| SX 25 | 0.00 | 0.01 | 0.01 | 0.00 | −0.01 | −0.01 | −0.01 | 0.00 |
| SX 26 | 0.08 | 0.04 | 0.01 | 0.02 | 0.01 | 0.03 | 0.00 | 0.00 |
| SX 27 | 0.12 | 0.05 | 0.02 | 0.00 | 0.01 | 0.00 | 0.01 | 0.00 |
| SX 28 | 0.08 | 0.06 | 0.05 | 0.04 | 0.01 | 0.01 | 0.01 | 0.01 |
| SX 29 | 2.77 | 2.71 | 2.46 | 2.17 | 1.72 | 1.26 | 0.71 | 0.41 |

The OMP preparation demonstrates increased background noise at higher concentrations of serum.

### Example 5

### Comparison of Endpoints

A comparison of the 29 samples illustrates that the novel lysate preparation of the present invention has less background noise at higher concentrations of serum. Less background in the assay will result in fewer false positives and an overall more precise assay.

For further analysis of the novel assay as compared to the prior art OMP preparation, the end point titrations of the previous 29 samples were compared in the following table.

TABLE 1

| | Titration | Titration |
|---|---|---|
| | Endpoint of | Endpoint of |
| | Novel | Prior Art |
| Sample | Lysate | OMP |
| SX 1 | <1 K | 2 K |
| SX 2 | <1 K | <1 K |
| SX 3 | <1 K | 1 K |
| SX 4 | <1 K | 2 K |
| SX 5 | <1 K | 4 K |
| SX 6 | <1 K | 2 K |
| SX 7 | 64 K | 64 K |
| SX 8 | <1 K | <1 K |
| SX 9 | 1 K | 4 k |
| SX 10 | 64 K | 128 K |
| SX 11 | 4 K | 2 K |
| SX 12 | 128 K | 128 K |
| SX 13 | 64 K | 64 K |
| SX 14 | <1 K | 2 K |
| SX 15 | 1 K | 2 K |
| SX 16 | 128 K | 64 K |
| SX 17 | 4 K | 8 K |
| SX 18 | 128 K | 128 K |
| SX 19 | <1 K | <1 K |
| SX 20 | <1 K | 4 K |
| SX 21 | <1 K | 1 K |
| SX 22 | <1 K | <1 K |
| SX 23 | <1 K | 1 K |
| SX 24 | <1 K | <1 K |
| SX 25 | <1 K | <1 K |
| SX 26 | <1 K | 2 K |
| SX 27 | <1 K | 2 K |
| SX 28 | <1 K | 8 K |
| SX 29 | >128 K | >128 K |

The comparison of the two assays produced the unexpected result that the background noise of the novel lysate preparation of the present invention produced almost no background noise, thereby indicating far fewer false positives. Samples 1 to 6, all pre-bleeds from cats, were negative—as expected under the novel lysate of the present invention. However, Samples 1 to 6, when run under the OMP preparation, showed a positive result in 5 of 6 cases. Reference to samples 7 to 12 illustrates that as the concentration of the antigen increases the results from the novel lysate preparation and the OMP preparation are more comparable. This observation is further confirmed by samples 13 to 18. Samples 19 to 27, which were samples expected to be antibody negative to B. henselae, demonstrated positive results in 5 of 9 cases under the prior art OMP preparation, but demonstrated negative results in 9 of 9 cases under the novel lysate preparation.

Further false binding (false positive) under the prior art preparations was demonstrated by the negative serum well of sample 28. The prior art OMP preparation resulted in a positive reading whereas the novel lysate preparation of the present invention resulted in a negative reading as expected.

Accordingly, the novel lysate preparation of the present invention produces lower background and specific binding as compared to the prior art OMP preparation.

### Example 6

### Comparison at Low Dilutions

A further experiment was run wherein 3 plates (plates 1, 2, and 3) were coated with the B. henselae OMP preparation at 187 μg/ml in Carbonate buffer at pH 9.6 and another 3 plates (plates 4, 5, and 6) were coated with B. henselae lysate at a concentration of 1:2K in PBS at pH 7.4. The top halves of the plates were coated with antigen, while the bottom halves were coated with buffer-only as controls. All plates were coated overnight at 4° C., then washed 1 time with wash buffer. The plates were blocked with 200 μl/well serum diluent and incubated for one hour at 37° C. The plates were then washed once with wash buffer.

The serum samples 1 to 29 from above were serially diluted 4-fold in serum diluent on both the top and bottom halves of the plates, beginning at 1:100. The plates were incubated for 1 hour at 37° C., then washed 4 times. The secondary antibody conjugate (HRP-labeled goat anti-cat IgG), diluted 1:2K in serum diluent, was added and the plates incubated for 1 hour at 37° C. The plates were washed 4 times, and K⁺ P TMB substrate added. The reaction was stopped after 10 minutes with 100 μl per well 2M H₂SO₄ and read immediately at 450 minus 540 nm, as above. Tables 1, 2, and 3 below contain the raw data:

A. Raw Data

TABLE 1
Table 1 is raw data from an assay of the prior art OMP
preparation for samples 1-12.

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 0.674 | 0.473 | 0.444 | 0.292 | 0.510 | 0.526 | 1.976 | 0.399 | 0.342 | 1.840 | 0.460 | 1.805 |
| 1:400 | 0.484 | 0.217 | 0.215 | 0.418 | 0.341 | 0.337 | 2.233 | 0.136 | 0.142 | 2.107 | 2.295 | 2.297 |
| 1:1600 | 0.214 | 0.090 | 0.074 | 0.166 | 0.113 | 0.086 | 1.626 | 0.126 | 0.065 | 1.572 | 0.110 | 1.933 |
| 1:6400 | 0.063 | 0.035 | 0.022 | 0.051 | 0.039 | 0.044 | 0.873 | 0.025 | 0.014 | 0.954 | 0.026 | 1.236 |
| 1:100 | 0.010 | 0.018 | 0.016 | 0.012 | 0.013 | 0.007 | 0.011 | 0.015 | 0.010 | 0.010 | 0.009 | 0.019 |
| 1:400 | 0.005 | 0.010 | 0.009 | 0.027 | 0.005 | 0.004 | 0.005 | 0.007 | 0.005 | 0.007 | 0.005 | 0.010 |
| 1:1600 | 0.004 | 0.007 | 0.014 | 0.011 | 0.008 | 0.013 | 0.006 | 0.003 | 0.004 | 0.003 | 0.004 | 0.015 |
| 1:6400 | 0.004 | 0.006 | 0.007 | 0.007 | 0.009 | 0.005 | 0.006 | 0.004 | 0.023 | 0.004 | 0.004 | 0.017 |

TABLE 2
Table 2 is raw data from an assay of the prior art OMP
preparation for samples 13-24.

| | SX | SX | SX | SX | SX | SX | SX | SX | SX | SX | SX | SX |
|---|---|---|---|---|---|---|---|---|---|---|---|---|
| | 13 | 14 | 15 | 16 | 17 | 18 | 19 | 20 | 21 | 22 | 23 | 24 |
| 1:100 | 2.116 | 0.445 | 0.623 | 2.212 | 0.372 | 2.157 | 0.136 | 0.231 | 0.210 | 0.093 | 0.270 | 0.032 |
| 1:400 | 1.888 | 0.198 | 0.357 | 2.800 | 0.208 | 2.242 | 0.060 | 0.122 | 0.089 | 0.029 | 0.133 | 0.015 |
| 1:1600 | 1.197 | 0.091 | 0.139 | 1.611 | 0.107 | 1.998 | 0.029 | 0.074 | 0.028 | 0.020 | 0.036 | 0.014 |
| 1:6400 | 0.511 | 0.056 | 0.027 | 0.750 | 0.030 | 1.380 | 0.009 | 0.019 | 0.014 | 0.006 | 0.015 | 0.008 |
| 1:100 | 0.012 | 0.015 | 0.024 | 0.025 | 0.016 | 0.008 | 0.012 | 0.005 | 0.006 | 0.006 | 0.019 | 0.008 |
| 1:400 | 0.007 | 0.350 | 0.007 | 0.038 | 0.005 | 0.007 | 0.005 | 0.010 | 0.004 | 0.006 | 0.006 | 0.003 |
| 1:1600 | 0.004 | 0.005 | 0.009 | 0.020 | 0.013 | 0.032 | 0.007 | 0.009 | 0.004 | 0.003 | 0.039 | 0.004 |
| 1:6400 | 0.007 | 0.004 | 0.008 | 0.010 | 0.006 | 0.012 | 0.019 | 0.006 | 0.033 | 0.005 | 0.035 | 0.014 |

TABLE 3
Table 3 is raw data from an assay of the prior art
OMP preparation for samples 25-29.

| | SX | SX | SX | SX | SX |
|---|---|---|---|---|---|
| | 25 | 26 | 27 | 28 | 29 |
| 1:100 | 0.120 | 0.262 | 0.296 | 0.540 | 2.159 |
| 1:400 | 0.070 | 0.176 | 0.190 | 0.291 | 2.487 |
| 1:1600 | 0.023 | 0.070 | 0.067 | 0.094 | 2.412 |
| 1:6400 | 0.011 | 0.043 | 0.026 | 0.040 | 2.210 |
| 1:100 | 0.007 | 0.015 | 0.019 | 0.033 | 0.017 |
| 1:400 | 0.022 | 0.007 | 0.005 | 0.038 | 0.023 |
| 1:1600 | 0.005 | 0.004 | 0.005 | 0.026 | 0.008 |
| 1:6400 | 0.017 | 0.006 | 0.008 | 0.020 | 0.006 |

TABLE 4
Table 4 is raw data from an assay of the novel lysate
preparation for samples 1-12.

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 0.085 | 0.179 | 0.170 | 0.240 | 0.217 | 0.447 | 2.718 | 0.236 | 0.308 | 2.684 | 1.187 | 2.740 |
| 1:400 | 0.042 | 0.071 | 0.070 | 0.102 | 0.047 | 0.164 | 2.383 | 0.062 | 0.069 | 2.384 | 0.749 | 2.552 |
| 1:1600 | 0.014 | 0.028 | 0.021 | 0.030 | 0.020 | 0.048 | 1.682 | 0.025 | 0.020 | 1.820 | 0.273 | 2.150 |
| 1:6400 | 0.010 | 0.008 | 0.008 | 0.022 | 0.010 | 0.013 | 0.672 | 0.013 | 0.010 | 0.709 | 0.063 | 1.044 |
| 1:100 | 0.046 | 0.020 | 0.024 | 0.026 | 0.039 | 0.006 | 0.075 | 0.013 | 0.042 | 0.013 | 0.031 | 0.014 |
| 1:400 | 0.027 | 0.008 | 0.010 | 0.028 | 0.020 | 0.005 | 0.013 | 0.007 | 0.035 | 0.009 | 0.013 | 0.007 |
| 1:1600 | 0.006 | 0.005 | 0.005 | 0.086 | 0.009 | 0.016 | 0.006 | 0.005 | 0.010 | 0.005 | 0.008 | 0.015 |
| 1:6400 | 0.006 | 0.005 | 0.006 | 0.005 | 0.005 | 0.004 | 0.005 | 0.005 | 0.022 | 0.004 | 0.006 | 0.009 |

TABLE 5
Table 5 is raw data from an assay of the novel lysate
preparation for samples 13-24.

| | SX | SX | SX | SX | SX | SX | SX | SX | SX | SX | SX | SX |
|---|---|---|---|---|---|---|---|---|---|---|---|---|
| | 13 | 14 | 15 | 16 | 17 | 18 | 19 | 20 | 21 | 22 | 23 | 24 |
| 1:100 | 2.709 | 0.146 | 0.172 | 2.68 | 0.524 | 2.803 | 0.024 | 0.106 | 0.125 | 0.026 | 0.108 | 0.081 |
| 1:400 | 2.474 | 0.065 | 0.080 | 2.352 | 0.213 | 2.792 | 0.018 | 0.056 | 0.022 | 0.010 | 0.053 | 0.025 |
| 1:1600 | 1.576 | 0.024 | 0.022 | 1.490 | 0.060 | 2.176 | 0.005 | 0.022 | 0.007 | 0.006 | 0.011 | 0.011 |
| 1:6400 | 0.674 | 0.025 | 0.007 | 0.638 | 0.042 | 1.239 | 0.009 | 0.007 | 0.004 | 0.005 | 0.006 | 0.007 |
| 1:100 | 0.028 | 0.014 | 0.040 | 0.019 | 0.016 | 0.008 | 0007 | 0.005 | 0.007 | 0.005 | 0.011 | 0.014 |
| 1:400 | 0.019 | 0.007 | 0.089 | 0.044 | 0.016 | 0.006 | 0.006 | 0.005 | 0.005 | 0.004 | 0.006 | 0.009 |
| 1:1600 | 0.009 | 0.004 | 0.017 | 0.018 | 0.010 | 0.016 | 0.013 | 0.004 | 0.007 | 0.004 | 0.007 | 0.006 |
| 1:6400 | 0.006 | 0.005 | 0.007 | 0.032 | 0.005 | 0.005 | 0.007 | 0.025 | 0.043 | 0.006 | 0.005 | 0.009 |

TABLE 6
Table 6 is raw data from an assay of the novel lysate
preparation for samples 25-29

| | SX | SX | SX | SX | SX |
|---|---|---|---|---|---|
| | 25 | 26 | 27 | 28 | 29 |
| 1:100 | 0.033 | 0.112 | 0.051 | 0.057 | 2.752 |
| 1:400 | 0.017 | 0.057 | 0.028 | 0.036 | 2.702 |
| 1:1600 | 0.010 | 0.022 | 0.011 | 0.012 | 2.610 |
| 1:6400 | 0.008 | 0.009 | 0.007 | 0.022 | 2.179 |
| 1:100 | 0.005 | 0.032 | 0.016 | 0.023 | 0.033 |
| 1:400 | 0.005 | 0.005 | 0.006 | 0.036 | 0.027 |
| 1:1600 | 0.007 | 0.006 | 0.007 | 0.038 | 0.027 |
| 1:6400 | 0.008 | 0.007 | 0.006 | 0.008 | 0.011 |

C. Adjusted Values

The raw data values above were converted into adjusted values by subtracting the optical densities of the negative side (the buffer side) from the optical densities of the positive side.

The cut-off values were determined by taking the mean of the control (buffer) wells plus 3 standard deviations of the control wells. The cut-off value for samples 1-12 of the prior art OMP preparation was 0.025. The cut-off value for samples 12-24 of the prior art OMP preparation was 0.041. The cut-off value for samples 24-29 of the prior art OMP preparation was 0.039. The cut-off value for samples 1-12 of the novel lysate preparation of the present invention was 0.068. The cut-off value for samples 13-24 of the novel lysate of the present invention was 0.058. The cut-off value for samples 25-29 of the novel lysate preparation of the present invention was 0.053.

The following tables 1-6 correspond to tables 1-6 of the raw data of the optical densities of the lower concentrations.

TABLE 1
Prior art OMP preparation

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 0.66 | 0 46 | 0 43 | 0 58 | 0.50 | 0 52 | 1 97 | 0.38 | 0.33 | 1.83 | 0.45 | 1.79 |
| 1:400 | 0.48 | 0.21 | 0.21 | 0 39 | 0.34 | 0 33 | 2 23 | 0.13 | 0 14 | 2 10 | 2.29 | 2.29 |
| 1.1600 | 0.21 | 0 08 | 0 06 | 0 16 | 0.11 | 0.07 | 1.62 | 0.12 | 0.06 | 1.57 | 0.11 | 1.92 |
| 1:6400 | 0.06 | 0.03 | 0.02 | 0 04 | 0 03 | 0.04 | 0 87 | 0 02 | −0 01 | 0 95 | 0.02 | 1 22 |

TABLE 2
Prior art OMP preparation

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 2 10 | 0 43 | 0.60 | 2.19 | 0.36 | 2.15 | 0.12 | 0 23 | 0 20 | 0 09 | 0.25 | 0.02 |
| 1:400 | 1.88 | −0.15 | 0 35 | 2.76 | 0 20 | 2 24 | 0 06 | 0 11 | 0 09 | 0 02 | 0 13 | 0 01 |
| 1:1600 | 1.19 | 0 09 | 0 13 | 1.59 | 0 09 | 1.97 | 0.02 | 0.07 | 0.02 | 0 02 | 0.00 | 0.01 |
| 1:6400 | 0.50 | 0 05 | 0.02 | 0 74 | 0 02 | 1 37 | −0.01 | 0.01 | −0.02 | 0.00 | −0.02 | −0.01 |

TABLE 3
Prior art OMP preparation

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 0 11 | 0 25 | 0 28 | 0 51 | 2 14 | 2 09 | 0.31 | 1.75 | 1.57 | 1.77 | 0 55 | 0.80 |
| 1:400 | 0.05 | 0 17 | 0.19 | 0.25 | 2.46 | 1.92 | 0 12 | 1 23 | 1 26 | 1 47 | 0 34 | 0.49 |
| 1:1600 | 0 02 | 0 07 | 0 06 | 0.07 | 2 40 | 1 49 | 0.03 | 0.80 | 0.44 | 0.81 | 0 16 | 0.11 |
| 1:6400 | −0.01 | 0.04 | 0.02 | 0.02 | 2.20 | 0.62 | 0 02 | 0 27 | 0 16 | 0 23 | 0 03 | 0.02 |

TABLE 4
Novel lysate preparation

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 0.04 | 0 16 | 0 15 | 0 21 | 0 18 | 0 44 | 2 64 | 0 22 | 0 27 | 2.67 | 1.16 | 2.73 |
| 1:400 | 0.02 | 0.06 | 0.06 | 0 07 | 0.03 | 0.16 | 2 37 | 0 06 | 0 03 | 2.38 | 0 74 | 2.55 |
| 1:1600 | 0 01 | 0 02 | 0.02 | −0.06 | 0.01 | 0 03 | 1 68 | 0 02 | 0 01 | 1.82 | 0 27 | 2.14 |
| 1:6400 | 0.00 | 0.00 | 0.00 | 0.02 | 0.01 | 0.01 | 0 67 | 0.01 | −0.01 | 0 71 | 0.06 | 1.04 |

TABLE 5
Novel lysate preparation

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 2 68 | 0 13 | 0 13 | 2.66 | 0 51 | 2 80 | 0 02 | 0 10 | 0 12 | 0 02 | 0.10 | 0.07 |
| 1:400 | 2.46 | 0 06 | −0.01 | 2.31 | 0.20 | 2 79 | 0 01 | 0 05 | 0.02 | 0 01 | 0 05 | 0 02 |
| 1.1600 | 1.57 | 0.02 | 0 00 | 1 47 | 0.05 | 2 16 | −0 01 | 0 02 | 0 00 | 0.00 | 0.00 | 0 01 |
| 1.6400 | 0.67 | 0 02 | 0.00 | 0.61 | 0.04 | 1 23 | 0.00 | −0 02 | −0 04 | 0 00 | 0 00 | 0 00 |

TABLE 6
Novel lysate preparation

| | SX 1 | SX 2 | SX 3 | SX 4 | SX 5 | SX 6 | SX 7 | SX 8 | SX 9 | SX 10 | SX 11 | SX 12 |
|---|---|---|---|---|---|---|---|---|---|---|---|---|
| 1:100 | 0.03 | 0.08 | 0.04 | 0.03 | 2.72 | 2 85 | 0 02 | 2 71 | 2 63 | 2 76 | 0.12 | 1.83 |
| 1:400 | 0 01 | 0 05 | 0 02 | 0 00 | 2 65 | 2.67 | 0 01 | 2.19 | 2 25 | 2.43 | 0 04 | 0.97 |
| 1:1600 | 0.00 | 0.02 | 0.00 | −0.03 | 2.58 | 2.27 | 0 00 | 1 20 | 1 21 | 1 66 | 0 02 | 0.34 |
| 1.6400 | 0 00 | 0 00 | 0.00 | 0.01 | 2.17 | 1.30 | 0.00 | 0.45 | 0.36 | 0 69 | 0.05 | 0 08 |

D. Example 6

The following table is a direct comparison of the OMP preparation and the novel lysate of the present invention at lower serum dilutions.

| | OMP | Lysate | OMP | Lysate | OMP | Lysate | OMP | Lysate |
|---|---|---|---|---|---|---|---|---|
| Dilution | 1:100 | 1:100 | 1:400 | 1:400 | 1:1600 | 1:1600 | 1:6400 | 1:6400 |
| SX 1 | 0 68 | 0 04 | 0 48 | 0.02 | 0 21 | 0 01 | 0.06 | 0.00 |
| SX 2 | 0.46 | 0 16 | 0.21 | 0 06 | 0 08 | 0.02 | 0.03 | 0 00 |
| SX 3 | 0 43 | 0 15 | 0.21 | 0 06 | 0.06 | 0 02 | 0 02 | 0.00 |
| SX 4 | 0.58 | 0 21 | 0 39 | 0 07 | 0.16 | −0 06 | 0.04 | 0.02 |
| SX 5 | 0 50 | 0.18 | 0 34 | 0.03 | 0 11 | 0.01 | 0 03 | 0 01 |
| SX 6 | 0.52 | 0 44 | 0 33 | 0.16 | 0 07 | 0 03 | 0 04 | 0.01 |
| SX 7 | 1 97 | 2.64 | 2.23 | 2.37 | 1 62 | 1.88 | 0 87 | 0 67 |
| SX 8 | 0 38 | 0 22 | 0 13 | 0 06 | 0 12 | 0 02 | 0 02 | 0.01 |
| SX 9 | 0 33 | 0.27 | 0 14 | 0 03 | 0 06 | 0 01 | −0.01 | −0 01 |
| SX 10 | 1 83 | 2 67 | 2.10 | 2.38 | 1 57 | 1.82 | 0 95 | 0.71 |
| SX 11 | 0 45 | 1.16 | 2.29 | 0 74 | 0.11 | 0 27 | 0 02 | 0 06 |
| SX 12 | 1.79 | 2.73 | 2.29 | 2 55 | 1 92 | 2 14 | 1 22 | 1.04 |
| SX 13 | 2 10 | 2 68 | 1 88 | 2.46 | 1.19 | 1 57 | 0.50 | 0 67 |
| SX 14 | 0.43 | 0.13 | −0.15 | 0 06 | 0.09 | 0 02 | 0.05 | 0.02 |
| SX 15 | 0 60 | 0 13 | 0 35 | −0 01 | 0 13 | 0 00 | 0 02 | 0.00 |
| SX 16 | 2.19 | 2.66 | 2.76 | 2.31 | 1 59 | 1 47 | 0.74 | 0.61 |
| SX 17 | 0 36 | 0 51 | 0 20 | 0.20 | 0.09 | 0.05 | 0 02 | 0.04 |
| SX 18 | 2.15 | 2.80 | 2 24 | 2 79 | 1 97 | 2 16 | 1.37 | 1.23 |
| SX 19 | 0 12 | 0 02 | 0 06 | 0 01 | 0 02 | −0.01 | −0.01 | 0.00 |
| SX 20 | 0 23 | 0.10 | 0 11 | 0 05 | 0 07 | 0 02 | 0.01 | −0.02 |
| SX 21 | 0.20 | 0 12 | 0 09 | 0.02 | 0.02 | 0 00 | −0 02 | −0.04 |
| SX 22 | 0 09 | 0.02 | 0 02 | 0 01 | 0 02 | 0 00 | 0.00 | 0 00 |
| SX 23 | 0 25 | 0 10 | 0.13 | 0.05 | 0.00 | 0 00 | −0 02 | 0.00 |
| SX 24 | 0 02 | 0.07 | 0 01 | 0 02 | 0 01 | 0 01 | −0.01 | 0 00 |
| SX 25 | 0 11 | 0 03 | 0.05 | 0 01 | 0 02 | 0.00 | −0 01 | 0.00 |
| SX 26 | 0 25 | 0.08 | 0 17 | 0 05 | 0 07 | 0 02 | 0.04 | 0.00 |
| SX 27 | 0.28 | 0 04 | 0.19 | 0.02 | 0.06 | 0.00 | 0 02 | 0.00 |
| SX 28 | 0 51 | 0.03 | 0 25 | 0 00 | 0 07 | −0 03 | 0 02 | 0 01 |
| SX 29 | 2.14 | 2.72 | 2.46 | 2.65 | 2.40 | 2.58 | 2 20 | 2 17 |

These adjusted values illustrate that the prior art OMP preparation demonstrates a greater binding (specific and non-specific) to the plate than does the novel lysate preparation of the present invention, thereby resulting in a greater number of false positives. Specifically, samples 1, 2, 3, 4, 5, 6, 8, 9, 14, 15, 19, 20, 21, 22, 23, 24, 25, 26, 27, and 28 resulted in a higher optical density values than the cut-off utilizing the prior art OMP preparation at dilutions ≧1:1600. These samples are expected to be negatives, as illustrated from the previous examples. However, the novel lysate of the present invention demonstrates all of these samples to be negative at dilutions ≧1:1600.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims. Further, all patents mentioned herein are herby incorporated by reference.

What I claim is:

1. A novel Bartonella antigen extraction for use in an enzyme-linked immunosorbent assay comprising the steps of:
(a) harvesting Bartonella bacteria cells;
(b) separating the Bartonella bacteria cells in a first separation;
(c) suspending the first separation in a first suspension;
(d) sonicating the first suspension; and,
(e) extracting the antigen in the supernatant.

2. The extraction of claim 1 further comprising separating the suspended Bartonella bacteria cells in a second separation and suspending the second separation in a second suspension before sonicating.

3. The extraction of claim 1 wherein the first suspension is in a detergent buffer.

4. The extraction of claim 2 wherein the second suspension is in a detergent buffer.

5. The extraction of claim 1 further comprising growing the Bartonella bacteria cells.

6. The extraction of claim 1 wherein the Bartonella is selected from the group consisting of Bartonella henselae, Bartonella quintana, Bartonella clarridgeiae, and Bartonella vinsonii.

7. The extraction of claim 1 wherein the step of separating the Bartonella bacteria cells are separated in a centrifuge.

8. The extraction of claim 1 wherein the step of separating the Bartonella bacteria cells further comprises vortexing.

9. The extraction of claim 1 wherein the step of suspending the pellet in a solution is in a buffered saline solution.

10. The extraction of claim 3 wherein the detergent solution is non-ionic.

11. The extraction of claim 1 further comprising utilizing the extracted supernatant in an immunoassay as a lysate.

12. The extraction of claim 11 wherein the immunoassay is an enzyme-linked immunosorbent assay.

13. The extraction of claim 11 wherein the lysate is a part of a diagnostic kit for detecting the presence of Bartonella bacteria antibodies selected from the group consisting of Bartonella henselae, Bartonella quintana, Bartonella clarridgeiae, and Bartonella vinsonii in a serum.

14. The extraction of claim 13 wherein the serum is taken from a cat.

15. A diagnostic kit for detecting the presence of antibodies to Bartonella in serum comprising:
a plate coated with a Bartonella lysate antigen; and,
an enzyme-labeled conjugate which binds to antibodies bound to the Bartonella lysate.

16. The diagnostic kit of claim 15 wherein the Bartonella lysate is prepared according to any one of claims 1 to 9.

17. The diagnostic kit of claim 15 further comprising a substrate which allows the visualization of the conjugate.

18. The kit of claim 16 wherein the substrate comprises an enzyme label.

19. The diagnostic kit of claim 18 wherein the substrate is a composition for providing a calorimetric, fluorimetric or chemiluminescent signal in the presence of the enzyme label.

20. The diagnostic kit of claim 15 further comprising a positive control.

21. The diagnostic kit of claim 15 further comprising a negative control.

22. The diagnostic kit of claim 15 further comprising a reader.
